(19) United States
(12) Patent Application Publication
(10) Pub. No.: US 2007/0202583 A1
(43) Pub. Date: Aug. 30, 2007

(54) Fermentation Process

(75) Inventors: Mads Peter Torry Smith, Raleigh, NC (US); Gregory Alan Crabb, Wake Forest, NC (US)

Correspondence Address: NOVOZYMES NORTH AMERICA, INC., 500 FIFTH AVENUE, SUITE 1600, NEW YORK, NY, 10110, US

(73) Assignee: Novozymes North America, Inc, Franklinton, NC (US)

(21) Appl. No.: 11/597,941

(22) PCT Filed: May 24, 2005
(86) PCT No.: PCT/US05/18421

§ 371 Date: Jan. 4, 2007

Related U.S. Application Data
(60) Provisional application No. 60/574,820, filed on May 27, 2004.

(51) Int. Cl. C12P 7/06; C12P 19/02

(52) U.S. Cl. 435/161; 435/105

(57) ABSTRACT

The present invention provides a process of fermenting starch-containing material, comprising i) fermenting starch-containing material by subjecting said material to a carbohy-drate-source generating enzyme and a fermenting microorganism, ii) reducing the particle size of the starch-containing material obtained from step i) and/or liquefying the starch-containing material obtained from step i), and iii) fermenting the liquefied material from step ii). The invention also relates to a process of producing a fermentation product including the fermentation process of the invention.

### FIELD OF THE INVENTION

The present invention relates to fermentation processes for producing fermentation products, such as ethanol, from starch-containing material.

### BACKGROUND OF THE INVENTION

Fermentation processes are used for making a vast number of commercial products, including alcohols (e.g., ethanol, methanol, butanol); organic acids (e.g., citric acid, acetic acid, itaconic acid, lactic acid, gluconic acid); ketones (e.g., acetone); amino acids (e.g., glutamic acid); gases (e.g., H₂ and CO₂), and more complex compounds, including, for example, antibiotics (e.g., penicillin and tetracycline); enzymes; vitamins (e.g., riboflavin, B₁₂, beta-carotene); hormones, and other compounds which are difficult to produce synthetically. Fermentation processes are also commonly used in the consumable alcohol (e.g., beer and wine), dairy (e.g., in the production of yogurt and cheese), leather, and tobacco industries.

There is a need for further improvement of fermentation processes and for improved processes which include a fermentation step.

### SUMMARY OF THE INVENTION

The present invention provides improved fermentation processes for producing a fermentation product, such as ethanol, from a starch-containing starting material. A fermentation process of the invention utilizes the starch-containing material more efficiently than a traditional fermentation process and/or boosts the fermentation rate.

In the first aspect the invention relates to a process of fermenting starch-containing material, which process comprises:
i) fermenting said starch-containing material by subjecting said material to a fermenting microorganism,
ii) reducing the particle size of the starch-containing material obtained from step i) and/or liquefying the starch-containing material obtained from step i), and
iii) fermenting.

In an embodiment the starch-containing material has been subjected to a carbohydrate-source generating enzyme prior to and/or simultaneously with fermentation step i)

The starch-containing material may preferably be mash, especially corn mash or un-gelatinized starch.

According to the second aspect the invention relates to a process for producing a fermentation product from starch-containing material, comprising
(a) reducing the particle size of starch-containing material;
(b) liquefying the material obtained in step (a);
(c) subjecting the liquefied material obtained in step (b) to a fermentation process of the invention.

According to a third aspect the invention relates to a process for producing a fermentation product from starch-containing material, comprising
(a) reducing the particle size of starch-containing material;
(b) subjecting the material in step (a) to a fermentation process of the invention in the presence of an acid alpha-amylase.
   The fermentation product may optionally be recovered, preferably by distillation. In a preferred embodiment the fermentation product is ethanol.

### BRIEF DESCRIPTION OF THE DRAWING

FIG. 1 shows an integral fermentation-liquefaction system suitable for carrying out the invention.

FIG. 2 shows the conversion percentage of residual starch containing material collected in a 2800 micro meter mesh sieve after initial SSF subjected to additional milling and/or liquefaction followed by additional SSF.

### DETAILED DESCRIPTION OF THE INVENTION

The present invention provides improved fermentation processes which are suitable for producing fermentation products, especially ethanol, from a starch-containing starting material.

Initially in fermentation processes fermentable sugar(s), i.e., DP₁₋₃ sugar(s), are released faster than the fermenting microorganism in question can metabolize them into the desired fermentation product. In other words, the fermentable sugar uptake rate of the fermenting microorganism is lower than the enzymatic sugar release rate. Over a period of time —after initiation of fermentation—the concentration of the fermenting microorganism increases and the sugar uptake rate becomes greater than the enzymatic sugar release rate. Because the remaining substrate fraction (i.e., residual starch fraction) becomes more and more difficult to hydrolyze for the enzyme(s) the release of fermentable sugar(s) decreases. This results in a decreased fermentation rate.

According to the present invention the overall fermentation performance decrease is prevented or at least reduced. This is accomplished by milling and/or liquefying the fermentation medium after a period of time or after fermentation as an integral part of the fermentation process.

The inventors have found that improved conversion rates of starch present in corn mash may be attained by securing that starch found in corn mash is made accessible to enzymatic degradation. Residual starch measured at the end of the fermentation seems to be inaccessible to enzymatic attack. It has been found that corn mash contains large particles that are inaccessible for enzymatic attack due to inadequate milling/liquefaction. Example 1 shows that large inaccessible starch particles in corn mash (after SSF) are made accessible by additional size reduction and/or liquefaction.

Thus, in the first aspect the invention concerns a fermentation process comprising:
i) fermenting starch-containing material by subjecting said material to a fermenting microorganism,
ii) reducing the particle size of the starch-containing material obtained from step i) and/or liquefying the starch-containing material obtained from step i), and
iii) fermenting.

In preferred embodiments of the invention the starch-containing material has been subjected to a carbohydrate-source generating enzyme prior to fermentation step i), simultaneously with fermentation step i), or prior to and simultaneously with fermentation step i).

The fermentation process of the invention may be a batch fermentation process carried out in a fermentation tank or the like. According to preferred embodiments of the invention a partial or especially the entire fermentation medium volume is liquefied over a period of time after initial fermentation has taken place. It is to be understood that the starch-containing particles in the fermentation medium volume to is to be reduced in size and/or liquefied in step ii) may be the entire or a fractionated part of the initially fermented starch-containing material from step i), e.g., a liquid fraction containing enzymes and/or a fraction containing the remaining starch-containing material. The additional step(s) is(are) preferably initiated at the time when the fermenting microorganism has consumed significantly all readily available fermentable sugars. In one embodiment of the invention the additional liquefaction step is initiated when the concentration of DP₁₋₃ sugars, preferably glucose and/or maltose, in the fermentation medium reaches below 15 g/L fermentation medium, preferably below 10 g/L, even more preferred below 5 g/L. In another embodiment the additional step(s) is(are) initiated when the fermentation product production rate has decreased to below 85%, preferably below 75%, even more preferred 65% of the maximal rate.

The additional step(s) (i.e. step ii) may be carried out by subjecting a side-stream of the initially fermented material to particle size reduction and/or liquefaction at elevated temperatures for a period of time and thereafter saccharifying and/or fermenting said material further, preferably as a SSF step. In an embodiment additional liquefaction is carried out by (slowly) transferring the fermentation medium to and/or through a liquefaction holding tank, where the additional particle size reduction is carried out and/or liquefaction is carried out at typically between 40 and 80° C., preferably between 50-70° C. for 1 to 60 minutes, preferably for between 20 to 40 minutes, such as around 30 minutes. In an embodiment an effective amount of alpha-amylase is added. The alpha-amylase may be any of the alpha-amylases mentioned below in the section “Alpha-Amylases”. In a preferred embodiment the alpha-amylase is of bacterial origin, preferably a Bacillus alpha-amylase, or a fungal alpha-amylase, preferably an acid Aspergillus alpha-amylase. After step ii) the material is re-cycled or introduced into the same or an additional fermentation tank. As the fermenting microorganism might be inactivated (killed) during the additional step(s) (e.g. due to elevated temperatures) the period of time for treating the entire fermentation volume should be at least as long as the doubling time of the fermenting microorganism. In the case where the fermenting microorganism is yeast this typically means that the entire fermentation volume can be additionally liquefied in 12 hours or more. The exact time period depends on the fermenting microorganism in question and the starting material. A skilled person in the art can based on the fermenting microorganism's doubling time and the starting material easily determine the period of time suitable for additional liquefaction of the entire fermentation volume.

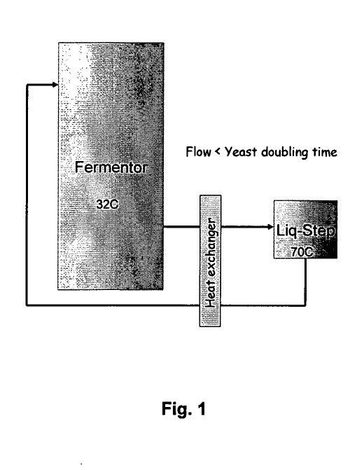

FIG. 1 illustrates an example of an integral fermentation-liquefaction system. It should be understood that FIG. 1 only illustrates one example of a system suitable for carrying out a process of the present invention. It is to be understood that a remaining starch-containing material after initial fermentation may also be separated, the particles reduced in size and/or liquefied using other means well know to the skilled person in the art.

During initial fermentation in step i) the fermenting microorganism consumes and converts easily accessible fermentable sugars into desired fermentation product. This ensures that essentially no fermentable sugars are lost or wasted due to the heating/elevated temperatures during the additional step ii). The additional step ii) may lead to increased fermentation rates, shorter fermentation times, higher starch/ethanol yields, i.e., higher conversion percentage, and/or less residual starch.

Fermentation

“Fermentation” refers to any fermentation process comprising a fermentation step. A fermentation process of the invention includes, without limitation, fermentation methods or processes used to produce alcohols (e.g., ethanol, methanol, butanol); organic acids (e.g., citric acid, acetic acid, itaconic acid, lactic acid, gluconic acid); ketones (e.g., acetone); amino acids (e.g., glutamic acid); gases (e.g., H₂ and CO₂); antibiotics (e.g., penicillin and tetracycline); enzymes; vitamins (e.g., riboflavin, B₁₂, beta-carotene); and hormones. Fermentation processes also include fermentation processes used in the consumable alcohol industry (e.g., beer and wine), dairy industry (e.g., fermented dairy products), leather industry and tobacco industry.

The process of the present invention may be used in combination with a saccharification process, e.g. SSF, in which additional enzymatic activities, such as esterase, such as lipase and/or cutinase, phytase, laccase, cellulase, xylanase, alpha-amylase, glucoamylase, glucosidase, protease, cellobiase, or mixtures thereof, may be used in processing the substrate, e.g., a starch substrate.

In yet another preferred embodiment, the fermentation process of the invention is used for the production of ethanol.

Fermentation Medium

“Fermentation medium” refers to the environment in which the fermentation is carried out and which includes the fermentation substrate, that is, the carbohydrate source that is metabolized by the fermenting microorganism(s). The fermentation medium, including fermentation substrate and other raw materials used in the fermentation process of the invention may be processed, e.g., by milling and/or liquefaction or other desired processes prior to the fermentation. Accordingly, the fermentation medium can refer to the medium before or after the fermenting microorganism(s) is(are) added, such as, the medium in or resulting from a liquefaction step, as well as the medium which comprises the fermenting microorganisms, such as, the medium used in a simultaneous saccharification and fermentation process (SSF).

Fermenting Microorganism

“Fermenting microorganism” refers to any organism, including bacterial and fungal organisms, suitable for use in a desired fermentation process. Especially suitable fermenting microorganisms are according to the invention able to ferment, i.e., to convert, sugars, such as DP₁₋₃ sugars, especially glucose or maltose, directly or indirectly into the desired fermentation product. Examples of fermenting organisms include fungal organisms, such as yeast. Preferred yeast includes strains of Saccharomyces spp., and in particular Saccharomyces cerevisiae. Commercially available yeast include, e.g., RED STAR®/Lesaffre Ethanol Red (available from Red Star/Lesaffre, USA), SUPERSTART (available from Alltech), GERT STRAND (available from Gert Strand AB, Sweden), FALI yeast (available from Fleischmann's Yeast, USA), and FERMIOL (available from DSM Specialties).

In ethanol production, the fermenting organism is preferably yeast, which is applied to the mash. Preferred yeast is derived from Saccharomyces spp., more preferably, from Saccharomyces cerevisiae. The yeast is applied to the starting material and the fermentation is ongoing for 24-96 hours, such as typically 35-60 hours. The temperature is generally between 26-34° C., in particular about 32° C., and the pH is generally from pH 3-6, preferably around pH 4-5. Yeast cells are preferably applied in amounts of 10⁵ to 10¹², preferably from 10⁷ to 10¹⁰, especially 5×10⁷ viable yeast count per ml of fermentation medium. During the ethanol producing phase the yeast cell count should preferably be in the range from 10⁷ to 10¹⁰, especially around 2×10⁵. Further guidance in respect of using yeast for fermentation can be found in, e.g., “The alcohol Textbook” (Editors K. Jacques, T. P. Lyons and D. R. Kelsall, Nottingham University Press, United Kingdom 1999), which is hereby incorporated by reference. Other fermentation organisms include Escherichia coli and Zymomonas mobilis.

Fermentation Substrate

Any suitable substrate or raw material may be used according to the present invention. The substrate is generally selected based on the desired fermentation product and the process employed, as is well known in the art. Examples of substrates suitable for use in the processes of present invention, include starch-containing plant materials, such as tubers, roots, whole grains, corns, cobs, wheat, barley, rye, milo or cereals, sugar-containing raw materials, such as molasses, fruit materials, sugar, cane or sugar beet, potatoes, and cellulose-containing materials, such as wood or plant residues. The term “liquefied mash” includes any of the above raw materials, which have been subjected to liquefaction using any method known in the art. Preferred is enzymatically mash, especially liquefied corn mash, and un-gelatinized starch (i.e., uncooked starch).

Carbohydrate-Source Generating Enzyme

The term “carbohydrate-source generating enzyme” includes glucoamylase (being a glucose generator), and beta-amylase and maltogenic amylase (being maltose generators). Other enzymes producing other carbohydrates suitable for the fermenting microorganism in question are also contemplated according to the invention. A carbohydrate-source generating enzyme is capable of providing energy to the fermenting microorganism(s) used in the process of the invention and/or may be converting directly or indirectly to the desired fermentation product, such as ethanol. The carbohydrate-source generating enzyme may be a mixture of enzymes falling within the definition. Especially contemplated mixtures are mixtures of at least glucoamylase and alpha-amylase, especially acid amylase, even more preferred fungal acid alpha-amylase. The ratio between acid fungal alpha-amylase activity (AFAU) per glucoamylase activity (AGU) (AFAU per AGU) may in one embodiment of the invention be at least 0.1, in particular at least 0.16, such as in the range from 0.12 to 0.50 or even higher such as between 0.5 and 1.0.

Examples of contemplated glucoamylases, alpha-amylases and beta-amylases are set forth in the sections below.

It is to be understood that the enzymes used according to the invention should be added in effective amounts. Effective amount can easily be determined by a person skilled in the art.

Glucoamylase

A glucoamylase used according to the invention may be derived from any suitable source, e.g., derived from a microorganism or a plant. Preferred glucoamylases are of fungal or bacterial origin, selected from the group consisting of Aspergillus glucoamylases, in particular A. niger G1 or G2 glucoamylase (Boel et al. (1984), EMBO J. 3 (5), p. 1097-1102), or variants thereof, such as disclosed in WO 92/00381, WO 00/04136 add WO 01/04273 (from Novozymes, Denmark); the A. awamori glucoamylase (WO 84/02921), A. oryzae (Agric. Biol. Chem. (1991), 55 (4), p. 941-949), or variants or fragments thereof.

Other Aspergillus glucoamylase variants include variants to enhance the thermal stability: G137A and G139A (Chen et al. (1996), Prot. Eng. 9, 499-505); D257E and D293E/Q (Chen et al. (1995), Prot. Engng. 8, 575-582); N182 (Chen et al. (1994), Biochem. J. 301, 275-281); disulphide bonds, A246C (Fierobe et al. (1996), Biochemistry, 35, 8698-8704; and introduction of Pro residues in position A435 and S436 (Li et al. (1997), Protein Engng. 10, 1199-1204. Other glucoamylases include Corticium rolfsii glucoamylase (U.S. Pat. No. 4,727,046) also referred to as Althelia rolfsii glucoamylase, Talaromyces glucoamylases, in particular, derived from Talaromyces emersonii (WO 99/28448), Talaromyces leycettanus (U.S. Pat. No. Re. 32,153), Talaromyces duponti, Talaromyces thermophilus (U.S. Pat. No. 4,587,215). Bacterial glucoamylases contemplated include glucoamylases from the genus Clostridium, in particular C. thermoamylolyticum (EP 135,138), and C. thermohydrosulfuricum (WO 86/01831).

Commercially available compositions comprising glucoamylase include AMG 200L; AMG 300 L; SAN™ SUPER, SAN™ EXTRA L, SPIRIZYME™ PLUS, SPIRIZYME™ FUEL, SPIRIZYME™ B4U and AMG™ E (from Novozymes A/S); OPTIDEX™ 300 (from Genencor Int.); AMIGASE™ and AMIGASE™ PLUS (from DSM); G-ZYME™ G900, G-ZYME™ and G990 ZR (from Genencor Int.).

Glucoamylases may in an embodiment be added in an amount of 0.02-20 AGU/g DS, preferably 0.1-10 AGU/g DS, such as 0.4-4 AGU/g DS, such as around 2 AGU/g DS.

Alpha-Amylase

According to the invention preferred alpha-amylases are of fungal or bacterial origin.

More preferably, the alpha-amylase is a Bacillus alpha-amylase, such as, derived from a strain of B. licheniformis, B. amyloliquefaciens, B. sultilis and B. stearothermophilus. Other alpha-amylases include alpha-amylase derived from a strain of the Bacillus sp. NCIB 12289, NCIB 12512, NCIB 12513 or DSM 9375, all of which are described in detail in WO 95/26397, and the alpha-amylase described by Tsukamoto et al., Biochemical and Biophysical Research Communications, 151 (1988), pp. 25-31.

The Bacillus alpha-amylase may also be a variant and/or hybrid, especially one described in any of WO 96/23873, WO 96/23874, WO 97/41213, WO 99/19467, WO 00/60059, and WO 02/10355 (all documents hereby incorporated by reference). Specifically contemplated alpha-amylase variants are disclosed in U.S. Pat. Nos. 6,093,562, 6,297,038, U.S. Pat. Nos. 6,187,576, and 6,867,031 (hereby incorporated by reference) and include Bacillus stearothermophilus alpha-amylase (BSG alpha-amylase) variants having a deletion of one to four amino acid in positions R179 to G182, preferably a double deletion disclosed in WO 1996/023873—see e.g., page 20, lines 1-10 (hereby incorporated by reference), preferably corresponding to delta(181-182) compared to the wild-type BSG alpha-amylase amino acid sequence set forth in SEQ ID NO:3 disclosed in WO 99/19467 or deletion of amino acids R179 and G180 using SEQ ID NO:3 in WO 99/19467 for numbering (which reference is hereby incorporated by reference). Even more preferred are Bacillus alpha-amylases, especially Bacillus stearothermophilus alpha-amylase, which have a double deletion corresponding to delta(181-182) and further comprises a N193F substitution (also denoted 1181*+G182*+N193F) compared to the wild-type BSG alpha-amylase amino acid sequence set forth in SEQ ID NO:3 disclosed in WO 99/19467.

A hybrid alpha-amylase specifically contemplated comprises 445 C-terminal amino acid residues of Bacillus licheniformis alpha-amylase (shown as SEQ ID NO: 4 in WO 99/19467) and the 37 N-terminal amino acid residues of the alpha-amylase derived from Bacillus amyloliquefaciens (shown as SEQ ID NO: 3 in WO 99/194676), with one or more, especially all, of the following substitution:

G48A+T49I+G107A+H156Y+A181T+N190F+I201F+A209V+Q264S (using the Bacillus licheniformis numbering). Also preferred are variants having one or more of the following mutations (or corresponding mutations in other Bacillus alpha-amylase backbones): H154Y, A181T, N190F, A209V and Q264S and/or deletion of two residues between positions 176 and 179, preferably deletion of E178 and G179 (using the SEQ ID NO: 5 numbering of WO 99/19467).

Other alpha-amylase includes alpha-amylases derived from a strain of Aspergillus, such as, Aspergillus oryzae and Aspergillus niger alpha-amylases. In a preferred embodiment, the alpha-amylase is an acid alpha-amylase. In a more preferred embodiment the acid alpha-amylase is an acid fungal alpha-amylase or an acid bacterial alpha-amylase. More preferably, the acid alpha-amylase is an acid fungal alpha-amylase derived from the genus Aspergillus. A commercially available acid fungal amylase is SP288 (available from Novozymes A/S, Denmark) or alternatively SP288 comprising a starch-binding domain.

In an embodiment, the alpha-amylase is an acid alpha-amylase. The term “acid alpha-amylase” means an alpha-amylase (E.C. 3.2.1.1) which added in an effective amount has activity at a pH in the range of 3.0 to 7.0, preferably from 3.5 to 6.0, or more preferably from 4.0-5.0.

One preferred acid fungal alpha-amylase is a Fungamyl-like alpha-amylase. In the present disclosure, the term “Fungamyl-like alpha-amylase” indicates an alpha-amylase which exhibits a high homology, i.e. more than 50%, more than 55%, more than 60%, more than 65%, more than 70%, more than 75%, more than 80%, more than 85% or even more than 90% homology (identity) to the amino acid sequence shown in SEQ ID No. 10 in WO96/23874.

Preferably the alpha-amylase is an acid alpha-amylase, preferably from the genus Aspergillus, preferably of the species Aspergillus niger. In a preferred embodiment the acid fungal alpha-amylase is the one from A. niger disclosed as “AMYA_ASPNG” in the Swissprot/TeEMBL. database under the primary accession no. P56271. Also variant of set acid fungal amylase having at least 70% identity, such as at least 80% or even at least 90% identity, such as at least 95% identity thereto is contemplated.

A preferred acid alpha-amylase for use in the present invention may be derived from a strain of B. licheniformis, B. amyloliquefaciens, and B. stearothermophilus.

Preferred commercial compositions comprising alpha-amylase include MYCOLASE from DSM (Gist Brochades), BAN™, TERMAMYL™ SC, FUNGAMYL™, LIQUOZYME™ X and SAN™ SUPER, SAN™ EXTRA L (Novozymes A/S) and CLARASE™ L-40,000, DEX-LO™, SPEYME FRED, SPEZYME™ AA, and SPEZYME™ DELTA AA (Genencor Int.), and the acid fungal alpha-amylase sold under the trade name SP 288 (available from Novozymes A/S, Denmark). The alpha-amylase may be added in amounts as are well-known in the art.

The bacterial alpha-amylase may be added in amounts as are well-known in the art. When measured in KNU units the alpha-amylase activity is preferably present in an amount of 0.5-5,000 NU/g of DS, in an amount of 1-500 KNU/kg of DS, or more preferably in an amount of 5-1,000 KNU/kg of DS, such as 10-100 KNU/kg DS.

When measured in AAU units the acid alpha-amylase activity is preferably present in an amount of 5-50,0000 AAU/kg of DS, in an amount of 500-50,000 AAU/kg of DS, or more preferably in an amount of 100-10,000 AAU/kg of DS, such as 500-1,000 AAU/kg DS. Fungal acid alpha-amylases are preferably added in an amount of 10-10,000 AFAU/kg of DS, in an amount of 500-2,500 AFAU/kg of DS, or more preferably in an amount of 100-1,000 AFAU/kg of DS, such as approximately 500 AFAU/kg DS.

Maltogenic Amylase

The amylase may also be a maltogenic alpha-amylase. A “maltogenic alpha-amylase” (glucan 1,4-alpha-maltohydrolase, E.C. 3.2.1.133) is able to hydrolyze amylose and amylopectin to maltose in the alpha-configuration. A maltogenic alpha-amylase from Bacillus stearothermophilus strain NCIB 11837 is commercially available from Novozymes A/S under the tradename NOVAMYL™. Maltogenic alpha-amylases are described in EP patent no. 120,693, U.S. Pat. Nos. 4,598,048, 4,604,355 and 6,162,628, which are hereby incorporated by reference. Preferably, the maltogenic alpha-amylase is used in a raw starch hydrolysis process, as described, e.g., in WO 95/10627, which is hereby incorporated by reference. When used as a maltose generating enzyme fungal alpha-amylases may be added in an amount of 0.001-1.0 AFAU/g DS, preferably from 0.002-0.5 AFAU/g DS, preferably 0.02-0.1 AFAU/g DS.

Beta-Amylase

At least according to the invention the a beta-amylase (E.C 3.2.1.2) is the name traditionally given to exo-acting maltogenic amylases, which catalyze the hydrolysis of 1,4-alpha-glucosidic linkages in amylose, amylopectin and related glucose polymers. Maltose units are successively removed from the non-reducing chain ends in a step-wise manner until the molecule is degraded or, in the case of amylopectin, until a branch point is reached. The maltose released has the beta anomeric configuration, hence the name beta-amylase.

Beta-amylases have been isolated from various plants and microorganisms (W. M. Fogarty and C. T. Kelly, Progress in Industrial Microbiology, vol. 15, pp. 112-115, 1979). These beta-amylases are characterized by having optimum temperatures in the range from 40° C. to 65° C. and optimum pH in the range from 4.5 to 7. A commercially available beta-amylase from barley is SPEZYME™ BBA 1500 from Genencor Int., USA.

Growth Stimulators.

In an embodiment of the process of the invention one or more growth stimulators are added to further improve the fermentation, and in particular, the performance of the fermenting organism, such as, rate enhancement and product yield. Preferred stimulators for growth include vitamins and minerals. Examples of vitamins include multivitamins, biotin, pantothenate, nicotinic acid, meso-inositol, thiamine, pyridoxine, para-aminobenzoic acid, folic acid, riboflavin, and Vitamins A, B, C, D, and E. See, e.g., Alfenore et al., Improving ethanol production and viability of Saccharomyces cerevisia by a vitamin feeding strategy during fed-batch process,” Springer-Verlag (2002), which is hereby incorporated by reference. Examples of minerals include minerals and mineral salts that can supply nutrients comprising P, K, Mg, S, Ca, Fe, Zn, Mn, and Cu.

Production of Enzymes

As disclosed above, the enzymes may be derived or obtained from any origin, including, bacterial, fungal, yeast or mammalian origin. The term “derived” means in this context that the enzyme may have been isolated from an organism where it is present natively, i.e. the identity of the amino acid sequence of the enzyme are identical to a native enzyme. The term “derived” also means that the enzymes may have been produced recombinantly in a host organism, the recombinant produced enzyme having either an identity identical to a native enzyme or having a modified amino acid sequence, e.g., having one or more amino acids which are deleted, inserted and/or substituted, i.e., a recombinantly produced enzyme which is a mutant and/or a fragment of a native amino acid sequence or an enzyme produced by nucleic acid shuffling processes known in the art. Within the meaning of a native enzyme are included natural variants. Furthermore, the term “derived” includes enzymes produced synthetically by, e.g., peptide synthesis. The term “derived” also encompasses enzymes which have been modified e.g. by glycosylation, phosphorylation, or by other chemical modification, whether in vivo or in vitro. The term “obtained” in this context means that the enzyme has an amino acid sequence identical to a native enzyme. The term encompasses an enzyme that has been isolated from an organism where it is present natively, or one in which it has been expressed recombinantly in the same type of organism or another, or enzymes produced synthetically by, e.g., peptide synthesis. With respect to recombinantly produced enzymes the terms “obtained” and “derived” refers to the identity of the enzyme and not the identity of the host organism in which it is produced recombinantly.

The enzymes may also be purified. The term “purified” as used herein covers enzymes free from other components from the organism from which it is derived. The term “purified” also covers enzymes free from components from the native organism from which it is obtained. The enzymes may be purified, with only minor amounts of other proteins being present. The expression “other proteins” relate in particular to other enzymes. The term “purified” as used herein also refers to removal of other components, particularly other proteins and most particularly other enzymes present in the cell of origin of the enzyme of the invention. The enzyme may be “substantially pure,” that is, free from other components from the organism in which it is produced, that is, for example, a host organism for recombinantly produced enzymes. In preferred embodiment, the enzymes are at least 75% (w/w) pure, more preferably at least 80%, at least 85%, at least 90%, at least 95%, at least 96%, at least 97%, at least 98%, or at least 99% pure. In another preferred embodiment, the enzyme is 100% pure.

The enzymes used in the present invention may be in any form suitable for use in the processes described herein, such as e.g. in the form of a dry powder or granulate, a non-dusting granulate, a liquid, a stabilized liquid, or a protected enzyme. Granulates may be produced, e.g. as disclosed in U.S. Pat. No. 4,106,991 and U.S. Pat. No. 4,661,452, and may optionally be coated by methods known in the art. Liquid enzyme preparations may, for instance, be stabilized by adding stabilizers such as a sugar, a sugar alcohol or another polyol, lactic acid or another organic acid according to established methods. Protected enzymes may be prepared according to the method disclosed in EP 238,216.

Fermentation Product Production Processes

The fermentation process of the invention can be used in any process of producing a fermentation product. A preferred application of the fermentation process of the invention described herein is in an ethanol production process (e.g., for use as a fuel or fuel additive).

Thus, in the second aspect the invention relates to a process for producing a fermentation product from starch-containing material, comprising
(a) reducing the particle size of starch-containing material;
(b) liquefying the product of step (a);
(c) subjecting the liquefied material obtained in step (b) to a fermentation process of the invention.
   Size Reduction of Starch-Containing Material

In the production of starch-based fermentation products, especially ethanol, according to the invention, the raw material, such as whole grain, preferably corn, is reduced in particle size, e.g., by milling in order to open up the structure and allow for further processing. Two processes are preferred according to the invention: wet milling and dry milling. Most used for, e.g., ethanol production is dry milling where the whole kernel is milled and used in the remaining part of the process. Wet milling may also be used and gives a good separation of germ and meal (starch granules and protein) and may advantageously, with a few exceptions, be applied at locations where there is a parallel production of, e.g., syrups. Both wet and dry milling processes are well known in the art. Other particle size reducing technologies such as emulsifying technology, rotary pulsation may also be used.

Liquefaction

For instance, ethanol production processes generally involves the steps of liquefaction, saccharification, fermentation, and optionally recovery, e.g., by distillation. In liquefaction step (b), e.g., milled (whole) grain raw material is broken down (hydrolyzed) into maltodextrins (dextrins). Hydrolysis may be carried out by acid treatment or enzymatically by alpha-amylase treatment, in particular a Bacillus alpha-amylase described above in the “alpha-amylase”-section. The raw material is in one preferred embodiment of the process of the invention milled whole grain. However, a side stream from starch processing may also be used. At the beginning of the process the starting material may be a slurry of from about 25 to about 45 wt-% milled whole grain, and water.

In an embodiment of the invention, enzymatic liquefaction is carried out as a three-step hot slurry process. The slurry is heated to between 60-95° C., preferably 80-85° C., and the enzyme(s) is(are) added to initiate liquefaction (thinning). Preferably at least an alpha-amylase is added. Then the slurry may in one embodiment be jet-cooked at a temperature between 95-140° C., preferably 105-125° C. to complete gelatinization of the slurry. Then the slurry is cooled to 60-95° C. and more enzyme(s) is (are) added to finalize hydrolysis (secondary liquefaction). The liquefaction process is carried out at around pH 4.5-6.5, in particular at a pH between 5 and 6. Milled and liquefied whole grains are known as mash.

The liquefaction step (b) may be performed in the presence of any alpha-amylase preferably one mentioned above in the section “Alpha-Amylase”. Preferred alpha-amylases are of fungal or bacterial origin. Bacillus alpha-amylases, variant and hybrids thereof, are specifically contemplated according to the invention. The alpha-amylase may be added in effective amounts well-known in the art.

The bacterial alpha-amylase may be added in amounts as are well-known in the art. When measured in KNU units the alpha-amylase activity is preferably present in an amount of 0.5-5,000 NU/g of DS, in an amount of 1-500 KNU/kg of DS, or more preferably in an amount of 5-1,000 KNU/kg of DS, such as 10-100 KNU/kg DS.

When measured in AAU units the acid alpha-amylase activity is preferably present in an amount of 0.005-500 AAU/g DS, in an amount of 0.500-50 AAU/g DS, or more preferably in an amount of 0.1-10 AAU/g of DS, such as 0.5-1 AAU/g DS.

Fungal alpha-amylases may be added in an amount of 0.001-1.0 AFAU/g DS, preferably from 0.002-0.5 AFAU/g DS, preferably 0.02-0.1 AFAU/g DS. Bacillus alpha-amylases may be added in effective amounts well known to the person skilled in the art.

Saccharification and Fermentation

To produce low molecular fermentable sugars, i.e., carbohydrate source that can be metabolized by a fermenting microorganism, such as yeast, the maltodextrin from the liquefaction step must be further hydrolyzed. Hydrolysis is performed enzymatically using a carbohydrate-source generating enzyme, such as preferably glucoamylase. Alternatively, e.g., alpha-glucosidases, beta-amylase, Maltogenic alpha-amylase or acid alpha-amylases may be used. According to the invention saccharification and fermentation are carried out simultaneously, i.e., as a SSF process. According to this aspect of the invention step (c) of the process for producing a fermentation product is carried out using the fermentation process of the invention as described above.

Recovery

In a preferred embodiment the fermentation product is recovered, e.g. by distillation using any method know in the art. The fermented material (mash) may be distilled to extract the fermentation product, in particular ethanol. The end product, obtained according to an ethanol production process of the invention may be used as, e.g., fuel ethanol; drinking ethanol, i.e., potable neutral spirits; or industrial ethanol.

In a third aspect the invention relates to a process for producing a fermentation product from starch-containing material, comprising
(a) reducing the particle size of starch-containing material;
(b) carrying out the fermentation process of the invention

The starch-containing material may according to this aspect of the invention be un-gelatinized starch (i.e. uncooked starch).

At the beginning of the process the starting material comprises about 25 to about 45 wt-% starch-containing material, e.g., milled whole grain and water.

Further details on how to carry out milling, liquefaction, saccharification, fermentation, distillation, and ethanol recovery are well known to the skilled person.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and de-scribed herein will become apparent to those skilled in the art from the foregoing description. Such modifications are also intended to fall within the scope of the appended claims. In the case of conflict, the present disclosure including definitions will control.

Various references are cited herein, the disclosures of which are incorporated by reference in their entireties. The present invention is further described by the following examples which should not be construed as limiting the scope of the invention.

Materials and Methods

Enzymes:

Bacterial Alpha-amylase A (BAAA): Bacillus stearothermophilus alpha-amylase variant with the mutations: 1181*+G182*+N193F disclosed in U.S. Pat. No. 6,187,576 and available on request from Novozymes A/S, Denmark.
Glucoamylase TN: Glucoamylase derived from Talaromyces emersonii and disclosed as SEQ ID NO: 7 in WO 99/28448 with side activity of Aspergillus niger glucoamylase and Aspergillus niger acid alpha-amylase and available on request from Novozymes A/S, Denmark.
   Yeast:
RED STAR available from Red Star/Lesaffre, USA
   Stock Solution for Iodine Method:
0.1 N I₂ dissolved 1.3 g I₂ and 2.0 g KI into 100 mL DI water
   Methods:
   Alpha-Amylase Activity (KNU)

The amylolytic activity may be determined using potato starch as substrate. This method is based on the break-down of modified potato starch by the enzyme, and the reaction is followed by mixing samples of the starch/enzyme solution with an iodine solution. Initially, a blackish-blue color is formed, but during the break-down of the starch the blue color gets weaker and gradually turns into a reddish-brown, which is compared to a colored glass standard.

One Kilo Novo alpha amylase Unit (KNU) is defined as the amount of enzyme which, under standard conditions (i.e. at 37° C.±0.05; 0.0003 M Ca²⁺; and pH 5.6) dextrinizes 5260 mg starch dry substance Merck Amylum solubile.

A folder EB-SM-0009.02/01 describing this analytical method in more detail is available upon request to Novozymes A/S, Denmark, which folder is hereby included by reference.

Determination of FAU Activity

One Fungal Alpha-Amylase Unit (FAU) is defined as the amount of enzyme, which breaks down 5.26 g starch (Merck Amylum solubile Erg. B.6, Batch 9947275) per hour based upon the following standard conditions:

| | |
|---|---|
| Substrate | Soluble starch |
| Temperature | 37° C. |
| pH | 4.7 |
| Reaction time | 7-20 minutes |

Determination of Acid Alpha-Amylase Activity (AFAU)

Acid alpha-amylase activity is measured in AFAU (Acid Fungal Alpha-amylase Units), which are determined relative to an enzyme standard.

The standard used is AMG 300 L (from Novozymes A/S, Denmark, glucoamylase wild-type Aspergillus niger G1, also disclosed in Boel et al. (1984), EMBO J. 3 (5), p. 1097-1102) and WO 92/00381). The neutral alpha-amylase in this AMG falls after storage at room temperature for 3 weeks from approx. 1 FAU/mL to below 0.05 FAU/mL.

The acid alpha-amylase activity in this AMG standard is determined in accordance with the following description. In this method, 1 AFAU is defined as the amount of enzyme, which degrades 5.260 mg starch dry matter per hour under standard conditions.

Iodine forms a blue complex with starch but not with its degradation products. The intensity of colour is therefore directly proportional to the concentration of starch. Amylase activity is determined using reverse colorimetry as a reduction in the concentration of starch under specified analytic conditions.
Alpha-amylase Starch+Iodine→Dextrins+Oligosaccharides 40° C., pH 2.5 Blue/violet t=23 sec. Decoloration

Standard conditions/reaction conditions: (per minute)

| | |
|---|---|
| Substrate: | Starch, approx. 0.17 g/L |
| Buffer: | Citate, approx. 0.03 M |
| Iodine (I₂): | 0.03 g/L |
| CaCl₂: | 1.85 mM |
| pH: | 2.50 ± 0.05 |
| Incubation temperature: | 40° C. |
| Reaction time: | 23 seconds |
| Wavelength: | lambda = 590 nm |
| Enzyme concentration: | 0.025A FAU/mL |
| Enzyme working range: | 0.01-0.0 AFAU/mL4 |

If further details are preferred these can be found in EB-SM-0259.02/01 available on request from Novozymes A/S, Denmark, and incorporated by reference.

Acid Alpha-Amylase Units (MU)

The acid alpha-amylase activity can be measured in AAU (Acid Alpha-amylase Units), which is an absolute method. One Acid Amylase Unit (AAU) is the quantity of enzyme converting 1 g of starch (100% of dry matter) per hour under standardized conditions into a product having a transmission at 620 nm after reaction with an iodine solution of known strength equal to the one of a color reference.

Standard conditions/reaction conditions:

| | |
|---|---|
| Substrate: | Soluble starch. Concentration approx. |
| | 20 g DS/L. |
| Buffer: | Citrate, approx. 0.13 M, pH = 4.2 |
| Iodine solution: | 40.176 g potassium iodide + 0.088 |
| | g iodine/L |
| City water | 15°-20° dH (German degree hardness) |
| pH: | 4.2 |
| Incubation temperature: | 30° C. |
| Reaction time: | 11 minutes |
| Wavelength: | 620 nm |
| Enzyme concentration: | 0.13-0.19 AAU/mL |
| Enzyme working range: | 0.13-0.19 AAU/mL |

The starch should be Lintner starch, which is a thin-boiling starch used in the laboratory as colorimetric indicator. Lintner starch is obtained by dilute hydrochloric acid treatment of native starch so that it retains the ability to color blue with iodine. Further details can be found in EP0140410B2, which disclosure is hereby included by reference.

Glucoamylase Activity (AGI)

Glucoamylase (equivalent to amyloglucosidase) converts starch into glucose. The amount of glucose is determined here by the glucose oxidase method for the activity determination. The method described in the section 76-11 Starch—Glucoamylase Method with Subsequent Measurement of Glucose with Glucose Oxidase in “Approved methods of the American Association of Cereal Chemists”. Vol.1-2 AACC, from American Association of Cereal Chemists, (2000); ISBN: 1-891127-12-8.

One glucoamylase unit (AGI) is the quantity of enzyme which will form 1 micromol of glucose per minute under the standard conditions of the method.

Standard conditions/reaction conditions:

| | |
|---|---|
| Substrate: | Soluble starch. |
| | Concentration approx. 16 g |
| | dry matter/L. |
| Buffer: | Acetate, approx. 0.04 M, |
| | pH = 4.3 |
| pH: | 4.3 |
| Incubation temperature: | 60° C. |
| Reaction time: | 15 minutes |
| Termination of the reac- | NaOH to a concentration of |
| tion: | approximately 0.2 g/L (pH˜9) |
| Enzyme concentration: | 0.15-0.55 AAU/mL |

The starch should be Lintner starch, which is a thin-boiling starch used in the laboratory as colorimetric indicator. Lintner starch is obtained by dilute hydrochloric acid treatment of native starch so that it retains the ability to color blue with iodine.

Glucoamylase Activity (AGU)

The Novo Glucoamylase Unit (AGU) is defined as the amount of enzyme, which hydrolyzes 1 micromole maltose per minute under the standard conditions 37° C., pH 4.3, substrate: maltose 23.2 mM, buffer: acetate 0.1 M, reaction time 5 minutes.

An auto-analyzer system may be used. Mutarotase is added to the glucose dehydrogenase reagent so that any alpha-D-glucose present is turned into beta-D-glucose. Glucose dehydrogenase reacts specifically with beta-D-glucose in the reaction mentioned above, forming NADH which is determined using a photometer at 340 nm as a measure of the original glucose concentration.

| AMG incubation: | |
|---|---|
| Substrate: | maltose 23.2 mM |
| Buffer: | acetate 0.1 M |
| pH: | 4.30 ± 0.05 |
| Incubation temperature: | 37° C. ± 1 |
| Reaction time: | 5 minutes |
| Enzyme working range: | 0.5-4.0 AGU/mL |

| Color reaction: | |
|---|---|
| GlucDH: | 430 U/L |
| Mutarotase: | 9 U/L |
| NAD: | 0.21 mM |
| Buffer: | phosphate 0.12 M; 0.15 M NaCl |
| pH: | 7.60 ± 0.05 |
| Incubation temperature: | 37° C. ± 1 |
| Reaction time: | 5 minutes |
| Wavelength: | 340 nm |

A folder (EB-SM-0131.02/01) describing this analytical method in more detail is available on request from Novozymes A/S, Denmark, which folder is hereby included by reference.

Determination of Homology (Identity)

In context of the invention the term polypeptide “homology” is understood as the degree of “identity” between two sequences indicating a derivation of the first sequence from the second. The homology may suitably be determined by means of computer programs known in the art such as GAP provided in the GCG program package (Program Manual for the Wisconsin Package, Version 8, August 1994, Genetics Computer Group, 575 Science Drive, Madison, Wis., USA 53711) (Needleman, S. B. and Wunsch, C. D., (1970), Journal of Molecular Biology, 48, 443-453. The following settings for amino acid sequence comparison are used: GAP creation penalty of 3.0 and GAP extension penalty of 0.1.

### EXAMPLES

### Examples 1

The purpose of the experiment was to investigate the accessibility of residual starch contained in corn mash particles after initial SSF.

Corn mash was sieved through 2800 micrometer sieve by washing with DI-water. The obtained starch containing particles were treated as follows:
1) One part was milled (wet) to smaller size particles.
2) Second part was milled and re-liquefied using Bacterial Alpha-Amylase A (BAAA) at a dose of 100 NU/g DS for 90 minutes at 85° C.
   A sample corresponding to the fraction which normally goes through a standard SSF fermentation was run as control.

All samples were adjusted to equal Total Solid (TS) values of 5% (w/w) and loaded into 15 mL snap-cap tube. Subsequently standard SSF fermentation (32° C., 65 hours) was conducted. After pH adjustment to pH 5 Glucoamylase TN was added (0.5 AGU/g DS) and the tubes were inoculated with 0.04 mL/g mash of 20 hour yeast propagate (RED STAR®) prepared on corn mash. Experiments were run in three replicates and controls were included in the fermentation. The fermentations were monitored by HPLC analysis of sugars and fermentation products.

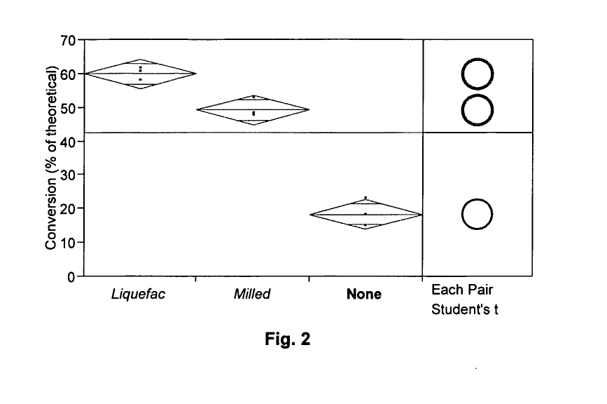

The result of the experiments are show in FIG. 2 showing that a significant portion of the corn mash—already subjected to SSF—is inaccessible unless the particles are subjected to additional milling and/or re-liquefaction. Theoretical level of corn mash is assumed to be 70% w/w for calculations. The circles in FIG. 2 were made using Student's test. Circles that do not overlap represent significant (alpha=0.05) different values.

1-31. (canceled)

32. A process of fermenting starch-containing material comprising:
(a) fermenting a starch-containing material by subjecting the material to a fermenting microorganism;
(b) liquefying the starch-containing material obtained in step (a); and
(c) fermenting.

33. The process of claim 32, wherein the particle size of the starch-containing material obtained in step (a) is reduced.

34. The process of claim 32, wherein the starch-containing material has been subjected to a carbohydrate-source generating enzyme prior to and/or simultaneously with step (a).

35. The process of claim 32, wherein the starch-containing material is mash or un-gelatinized starch.

36. The process of claim 32, wherein the starch-containing material is un-gelatinized starch that is simultaneously saccharified and fermented.

37. The process of claim 32, wherein step (b) is carried out by subjecting a side stream of the material obtained in step (a) to liquefaction for a period of time and thereafter fermenting the material.

38. The process of claim 37, wherein the liquefied material obtained in step (b) is recycled to step (a).

39. The process of claim 32, wherein the time in step (b) for liquefying the entire volume of the starch-containing material obtained in step (a) is longer than the doubling time of the fermenting microorganism of step (a).

40. The process of claim 39, wherein the time in step (b) for liquefying the entire volume of starch-containing material is 12 hours or more.

41. The process of claim 32, wherein the liquefaction in step (b) is carried out at a temperature between 40 and 80° C.

42. The process of claim 32, wherein the liquefaction in step (b) is carried out for between 1 and 60 minutes.

43. The process of claim 32, wherein an alpha-amylase is added in step (b).

44. The process of claim 32, wherein the liquefaction in step (b) is initiated when the DP₁₋₃ sugar concentration in the fermentation medium reaches below 15 g/L.

45. The process of claim 32, wherein the liquefaction in step (b) is initiated when the product production rate has decreased to below 65% of the maximal rate.

46. The process of claim 32, wherein the carbohydrate-source generating enzyme(s) is(are) selected from the group consisting of glucoamylase, beta-amylase, maltogenic alpha-amylase and acid alpha-amylase, or mixtures thereof.

47. The process of claim 32, wherein step (a) is part of a simultaneous saccharification and fermentation process.

48. The process of claim 32, wherein step (c) is carried out as a simultaneous saccharification and fermentation process.

49. The process of claim 32, wherein the fermenting microorganism in step (a) and/or (c) is a yeast.

50. The process of claim 32, wherein the fermentation is carried out at a temperature between about 25° C. to about 40° C.

51. The process of claim 32, further comprising recovering the fermentation product after step (c).

52. The process of claim 51, wherein the fermentation product is ethanol.

53. A process for producing a fermentation product from starch-containing material, comprising (a) reducing the particle size of starch-containing material;
(b) liquefying the product obtained in step (a); and
(c) subjecting the liquefied material obtained in step (b) to a fermentation process as defined in claim 32.

54. A process for producing a fermentation product from starch-containing material, comprising
(a) reducing the particle size of starch-containing material; and
(b) subjecting the material obtained in step (a) to a fermentation process defined in claim 32 in the presence of an acid alpha-amylase.
